(19) United States
(12) Patent Application Publication
(10) Pub. No.: US 2017/0266253 A1
(43) Pub. Date: Sep. 21, 2017

(54) ANTITHETICAL REGULATION OF ENDOTHELIAL ACE AND ACE2 BY BRG1-FOXM1 COMPLEX UNDERLIES PATHOLOGICAL CARDIAC HYPERTROPHY

(71) Applicant: Indiana University Research and Technology Corporation, Indianapolis, IN (US)

(72) Inventors: Ching-Pin Chang, Indianapolis, IN (US); Jin Yang, Carmel, IN (US)

(73) Assignee: Indiana University Research and Technology Corporation, Indianapolis, IN (US)

(21) Appl. No.: 15/500,161

(22) PCT Filed: Jul. 31, 2015
(86) PCT No.: PCT/US2015/043079

§ 371 Date: Jan. 30, 2017

Related U.S. Application Data
(60) Provisional application No. 62/031,450, filed on Jul. 31, 2014.

(51) Int. Cl. A61K 38/12

(52) U.S. Cl. CPC A61K 38/12

(57) ABSTRACT

Methods are disclosed herein for administering a FoxM1 inhibitor for preventing, treating, and/or reducing cardiac hypertrophy and/or cardiac failure. Particularly, the methods are directed to the use of a FoxM1 inhibitor to block the function of FoxM1-Brg1 complex, thereby reversing the ACE/ACE2 expression ratio such to protect the heart from hypertrophy and failure.

### CROSS-REFERENCE TO RELATED APPLICATION

This application claims priority from U.S. Provisional Application Ser. No. 62/031,450, filed Jul. 31, 2014, which is hereby incorporated by reference in its entirety.

### BACKGROUND OF THE DISCLOSURE

The field of the disclosure relates generally to inhibiting FoxM1 (Forkhead Box M1), thereby preventing and/or treating cardiac hypertrophy and failure in a subject. Particularly, in pathologically stressed hearts, FoxM1 and Brg1 (ATP-dependent helicase SMARCA4 (Switch/Sucrose nonfermentable related, matrix associated, actin dependent regulator of chromatin subfamily a, member 4)) are activated in cardiac endothelial cells. Brg1 and FoxM1 form a protein complex on angiotensin-converting enzyme (ACE) and angiotensin-converting enzyme 2 (ACE2) promoters and cooperate to simultaneously activate Ace and repress Ace2 express, leading to increased production of angiotensin II, causing cardiac hypertrophy and failure. The present disclosure has found that a FoxM1 inhibitor can block the function of FoxM1-Brg1 complex, reversing the ACE/ACE2 expression ratio to protect the heart from hypertrophy and failure.

Heart failure is the leading cause of death with a mortality rate of ˜50% within 5 years of diagnosis. This disorder is generally preceded by pathological hypertrophy of heart muscle, and most heart failure studies focus on the response of cardiomyocytes to pathological stress. Much less is known about how endothelial cells, which form a dense meshwork enclosing each single cardiomyocyte, and may modulate the latter's reaction to pathological insults and subsequent hypertrophy.

Heart function is regulated in part by angiotensin peptides, which have higher concentrations in the heart than in the circulation. Within the heart, greater than 90% of angiotensin I is synthesized locally, and greater than 75% of angiotensin II produced by enzymatic conversion of local cardiac angiotensin I (Ang I) to Ang II. Cardiac (coronary) endothelial cells are the primary source that produces angiotensin-converting enzymes (Ace and Ace2) to control angiotensin production. Ace and Ace2 are tethered to endothelial cell membrane or secreted into the interstitial space, where these enzymes process Ang I and II peptides. Biochemically, Ace converts the decapeptide Ang I (1-12) to octapeptide Ang II (1-10), while Ace2 degrades Ang II to form Ang-(1-7)14 and cleaves Ang I into Ang-(1-9). Functionally, Ang II is a potent stimulant of cardiac hypertrophy and fibrosis, whereas Ang-(1-7) and Ang-(1-9) counteract Ang II's cardiac effects to maintain heart function. When the heart is pathologically stressed, Ace is up-regulated with down-regulation of Ace2, tipping the balance to Ace dominance with enhanced Ang II and reduced Ang-(1-7) and (1-9) production. Such Ace/Ace2 perturbation contributes to the development of hypertrophy and heart failure. Inhibition of Ace or overexpression of Ace2 protects the heart from stress-induced failure; conversely, Ace2 knockout mice exhibit heart dysfunction. Therefore, Ace promotes cardiac pathology, whereas Ace2 inhibits cardiomyopathy. Balancing Ace/Ace2 is thus critical for maintaining heart function.

However, it is unclear how Ace and Ace2 expression is controlled by endothelial cells within the heart. Gene regulation requires control at the level of chromatin, which provides a dynamic scaffold to package DNA and dictates accessibility of DNA sequence to transcription factors. The present disclosure shows that Brg1, an essential ATPase subunit of the BAF chromatin-remodeling complex, is activated by pathological stress within the endothelium of mouse hearts to control Ace and Ace2 expression. Brg1 complexes with the forkhead box transcription factor FoxM1 that has both transactivating and repressive domains to bind to Ace and Ace2 promoters to simultaneously activate Ace and repress Ace2 transcription. Mice with endothelial Brg1 deletion or with FoxM1 inhibition or genetic disruption show resistance to stress-induced Ace/Ace2 switch, cardiac hypertrophy, and heart dysfunction. In human hypertrophic hearts, Brg1 and FoxM1 are also highly activated, and their activation correlates strongly with Ace/Ace2 ratio and the disease severity, indicating a conserved endothelial mechanism for human cardiomyopathy. Brg1 and FoxM1 are therefore essential endothelial mediators of cardiac stress. Given the lack of Ace2 drugs that limit full clinical exploitation of this pathway, targeting Brg1-FoxM1 complex may offer an alternative strategy for concurrent Ace and Ace2 control in heart failure therapy.

### BRIEF DESCRIPTION OF THE DISCLOSURE

The present disclosure is generally directed to the use of a FoxM1 inhibitor, and in particular, thiostrepton (see FIG. 1), to prevent, reduce, and treat hypertrophy and heart failure. Particularly, in pathologically stressed hearts, FoxM1 and Brg1 are activated in cardiac endothelial cells. FoxM1 cooperates with Brg1 to activate angiotensin-converting enzyme (ACE) and inhibit angiotensin-converting enzyme 2 (ACE2) expression, leading to increased production of angiotensin II, causing cardiac hypertrophy and failure. The present disclosure has found that a FoxM1 inhibitor can block the function of FoxM1-Brg1 complex, reversing the ACE/ACE2 expression ratio to protect the heart from hypertrophy and failure.

Accordingly, in one aspect, the present disclosure is directed to a method for treating cardiac hypertrophy in a subject in need thereof, the method comprising administering to the subject a FoxM1 inhibitor.

In another aspect, the present disclosure is directed to a method for treating cardiac failure in a subject in need thereof, the method comprising administering to the subject a FoxM1 inhibitor.

In another aspect, the present disclosure is directed to a method of modulating ACE/ACE2 enzyme ratio in a subject in need thereof, the method comprising administering to the subject a FoxM1 inhibitor.

### BRIEF DESCRIPTION OF THE DRAWINGS

FIG. 1 depicts the chemical structure of the FoxM1 inhibitor, Thiostrepton.

FIG. 2A depicts quantitative RT-PCR analysis of eNos, Et-1, Adamts1, Hdac7, Nrg1, ACE and ACE2 in the mice heart ventricles after sham or TAC operation as analyzed in Example 1. n=5 mice per group. P-value: Student's t-test. Error bar: SEM.

FIG. 2B depicts immunostaining of ACE in mice heart 7 days after sham operation. (scale bar, 20 μm). Arrows: interstitial space.

FIG. 2C depicts immunostaining of ACE in mice heart 7 days after TAC operation. (scale bar, 20 μm). Arrows: interstitial space.

FIG. 2D is a fluorescence micrograph depicting an overlay of co-immunostaining images of ACE2 (red channel), Pecam (green channel) and DAPI staining of nuclei (blue channel) in mice heart 7 days after sham operation. (scale bar, 10 μm).

FIG. 2E is a fluorescence micrograph depicting an overlay of co-immunostaining images of ACE2 (red channel), Pecam (green channel) and DAPI staining of nuclei (blue channel) in mice heart 7 days after TAC operation. (scale bar, 10 μm).

FIG. 2F is a fluorescence micrograph depicting an overlay of co-immunostaining images of Brg1 (red channel), Pecam (green channel) and DAPI staining of nuclei (blue channel) in mice heart 7 days after sham operation. (scale bar, 10 μm).

FIG. 2G is a fluorescence micrograph depicting an overlay of co-immunostaining images of Brg1 (red channel), Pecam (green channel) and DAPI staining of nuclei (blue channel) in mice heart 7 days after TAC operation. (scale bar, 10 μm).

FIG. 2H depicts a Western blot analysis of Brg1, ACE and ACE2 expression in mice hearts 7 days after sham or TAC operation.

FIG. 2I is a graph depicting quantitation of Brg1, ACE and ACE2 expression in mice hearts 7 days after sham or TAC operation. P-value: Student's t-test. Error bar: SEM.

FIG. 3A depicts β-galactosidase staining of SclCre^{ERT}; Rosa mice heart without tamoxifen treatment as analyzed in Example 2. (scale bar, 10 μm).

FIG. 3B depicts β-galactosidase staining of SclCre^{ERT}; Rosa mice heart with tamoxifen treatment as analyzed in Example 2. (scale bar, 10 μm). Arrows: endothelial cells.

FIG. 3C is a fluorescence micrograph depicting an overlay of co-immunostaining images of Brg1 (red channel), Pecam (green channel) and DAPI staining of nuclei (blue channel) in SclCre^{ERT}; Brg^{f/f} mice 7 days after TAC operation without tamoxifen treatment. (scale bar, 10 μm). Arrows: endothelial cell nuclei; Arrowheads: myocardial cell nuclei.

FIG. 3D is a fluorescence micrograph depicting an overlay of co-immunostaining images of Brg1 (red channel), Pecam (green channel) and DAPI staining of nuclei (blue channel) in SclCre^{ERT}; Brg^{f/f} mice 7 days after TAC operation with tamoxifen treatment. (scale bar, 10 μm). Arrows: endothelial cell nuclei; Arrowheads: myocardial cell nuclei.

FIG. 3E depicts photographs of whole hearts harvested 4 weeks after sham or TAC operation in control and SclCre^{ERT}; Brg^{f/f} mice treated with tamoxifen. (scale bar, 2 mm)

FIG. 3F depicts quantitation of ventricle—body weight ratio in control and SclCre^{ERT}; Brf^{f/f} mice 4 weeks after sham or TAC operation. Ctrl: control hearts. Mut: SclCre^{ERT}; Brg^{f/f} hearts. P-value: Student's t-test. Error bar: SEM.

FIG. 3G depicts quantitation of cardiomyocyte size in control and SclCre^{ERT}; Brg^{f/f} mice 4 weeks after sham or TAC operation.

FIG. 3H depicts trichrome staining of cardiac fibrosis in control mice 4 weeks after sham or TAC operation. (scale bar, 50 μm).

FIG. 3I depicts trichrome staining of cardiac fibrosis in SclCre^{ERT}; Brg^{f/f} 4 weeks after sham or TAC operation. (scale bar, 50 μm).

FIG. 3J depicts echocardiographic measurement of fractional shortening of the left ventricle after 4 weeks of TAC.

FIG. 3K depicts representative pressure volume loops taken after left-ventricular (LV) catheterization of control and SclCre^{ERT}; Brg^{f/f} mice 4 weeks after sham or TAC operation.

FIG. 3L depicts quantitation of left ventricular systolic pressure after 4 weeks of sham or TAC operation.

FIG. 3M depicts quantitation of ejection fraction (EF) after 4 weeks of sham or TAC operation.

FIG. 3N depicts quantitation of preload-adjusted maximum power after 4 weeks of sham or TAC operation.

FIG. 3O depicts quantitation of stroke volume (SV) after 4 weeks of sham or TAC operation.

FIG. 3P depicts quantitation of stroke work (SW) after 4 weeks of sham or TAC operation.

FIG. 3Q depicts quantitation of end systolic volume (ESV) after 4 weeks of sham or TAC operation.

FIG. 3R depicts quantitation of end diastolic volume (EDV) after 4 weeks of sham or TAC operation.

FIG. 3S depicts quantitation of Tau after 4 weeks of sham or TAC operation.

FIG. 3T depicts quantitation of end diastolic pressure (EDP) after 4 weeks of sham or TAC operation.

FIG. 3U depicts quantitation of cardiac output (CO) after 4 weeks of sham or TAC operation.

FIG. 4A depicts immunostaining of Pecam in control hearts 7 days after sham operation.

FIG. 4B depicts immunostaining of Pecam in control hearts 7 days after TAC operation.

FIG. 4C depicts immunostaining of Pecam in SclCre^{ERT}; Brg^{f/f} hearts 7 days after sham operation.

FIG. 4D depicts immunostaining of Pecam in SclCre^{ERT}; Brg^{f/f} hearts 7 days after TAC operation.

FIG. 4E depicts quantitation of vessels/cardiomyocyte in control and SclCre^{ERT}; Brg^{f/f} hearts after 2 weeks with sham or TAC operation. (scale bar, 10 μm).

FIG. 4F depicts WGA staining of heart tissue in control hearts after 4 weeks with sham operation.

FIG. 4G depicts WGA staining of heart tissue in control hearts after 4 weeks with TAC operation.

FIG. 4H depicts WGA staining of heart tissue in SclCre^{ERT}; Brg^{f/f} hearts after 4 weeks with sham operation.

FIG. 4I depicts WGA staining of heart tissue in SclCre^{ERT}; Brg^{f/f} hearts after 4 weeks with TAC operation.

FIG. 5A depicts quantitation of ACE, ACE2 and ACE/ACE2 in control and SclCre^{ERT}; Brg^{f/f} hearts after 2 weeks with sham or TAC operation. Ctrl: control hearts. Mut: SclCre^{ERT}; Brg^{f/f} hearts as analyzed in Example 3.

FIG. 5B depicts immunostaining of ACE in control hearts 7 days after sham operation. (scale bar, 10 μm).

FIG. 5C depicts immunostaining of ACE in control hearts 7 days after TAC operation. (scale bar, 10 μm).

FIG. 5D depicts immunostaining of ACE in SclCre^{ERT}; Brg^{f/f} hearts 7 days after sham operation. (scale bar, 10 μm).

FIG. 5E depicts immunostaining of ACE in SclCre^{ERT}; Brg^{f/f} hearts 7 days after TAC operation. (scale bar, 10 μm).

FIG. 5F depicts immunostaining of ACE2 in control hearts 7 days after sham operation. (scale bar, 10 μm).

FIG. 5G depicts immunostaining of ACE2 in control hearts 7 days after TAC operation. (scale bar, 10 μm).

FIG. 5H depicts immunostaining of ACE2 in SclCre^{ERT} ; Brg^{f/f} hearts 7 days after sham operation. (scale bar, 10 μm).

FIG. 5I depicts immunostaining of ACE2 in SclCre^{ERT}; Brg^{f/f} hearts 7 days after TAC operation. (scale bar, 10 μm).

FIG. 5J depicts Western blot analysis of ACE and ACE2 expression in control and SclCre^{ERT}; Brg^{f/f} hearts 7 days after sham or TAC operation.

FIG. 5K is a graph depicting quantitation of ACE and ACE2 expression in control and SclCre^{ERT}; Brg^{f/f} hearts 7 days after sham or TAC operation. P-value: Student's t-test. Error bar: SEM.

FIG. 5L depicts sequence alignment of the ACE locus from human and rat. Peak heights indicate degree of sequence homology. Black boxes (a1-a4) are regions of high sequence homology and were further analyzed by ChIP. Dark grey in regions a3, a2, and a1, promoter elements. Light grey, untranslated regions. Medium grey at region a4, transposons/simple repeats.

FIG. 5M depicts sequence alignment of ACE2 locus from mouse, human and rat. Peak heights indicate degree of sequence homology. Black boxes (b1-b5) are regions of high sequence homology and were further analyzed by ChIP.

FIG. 5N depicts ChIP-qPCR analysis of ACE promoter using antibodies against Brg1 (J1 antibody). P-value: Student's t-test. Error bar: SEM.

FIG. 5O depicts ChIP-qPCR analysis of ACE2 promoter using antibodies against Brg1 (J1 antibody). P-value: Student's t-test. Error bar: SEM.

FIG. 5P depicts luciferase reporter assays of the ACE (−2983bp to +174bp) and ACE2 (−7063bp to +786bp) proximal promoter in MCEC cells. P-value: Student's t-test. Error bar: SEM.

FIG. 6A depicts quantitative PCR analysis of FoxM1 expression in the mice heart ventricles after sham or TAC operation as analyzed in Example 4. n=4 mice per group.

FIG. 6B is a fluorescence micrograph depicting an overlay of co-immunostaining images of FoxM1 (red channel), Pecam (green channel) and DAPI staining of nuclei (blue channel) in mice heart 7 days after sham operation. (scale bar, 10 μm).

FIG. 6C is a fluorescence micrograph depicting an overlay of co-immunostaining images of FoxM1 (red channel), Pecam (green channel) and DAPI staining of nuclei (blue channel) in mice heart 7 days after TAC operation. (scale bar, 10 μm).

FIG. 6D depicts quantitation of ventricle—body weight ratio of mice treated with DMSO and thiostrepton after 4 weeks sham or TAC operation. Ctrl: DMSO. Thio: thiostrepton.

FIG. 6E depicts trichrome staining of cardiac fibrosis in mice treated with DMSO after 4 weeks sham or TAC operation. (scale bar, 20 um) Ctrl: DMSO. Thio: thiostrepton.

FIG. 6F depicts trichrome staining of cardiac fibrosis in mice treated with thiostrepton after 4 weeks sham or TAC operation. (scale bar, 20 μm) Ctrl: DMSO. Thio: thiostrepton.

FIG. 6G depicts echocardiographic measurement of fractional shortening of the left ventricle after 4 weeks of TAC. Ctrl: DMSO. Thio: thiostrepton.

FIG. 6H depicts Western blot analysis of ACE and ACE2 expression in the heart of the DMSO and thiostrepton treated mice 2 weeks after sham or TAC operation.

FIG. 6I is a graph depicting quantitation of ACE and ACE2 expression in the heart of the DMSO and thiostrepton treated mice 2 weeks after sham or TAC operation.

FIG. 6J is a fluorescence micrograph depicting an overlay of co-immunostaining images of FoxM1 (red channel), Pecam (green channel) and DAPI staining of nuclei (blue channel) in control mice 4 weeks after TAC operation with tamoxifen treatment. (scale bar, 10 μm). Arrows: endothelial cell nuclei; Arrowheads: myocardial cell nuclei.

FIG. 6K is a fluorescence micrograph depicting an overlay of co-immunostaining images of FoxM1 (red channel), Pecam (green channel) and DAPI staining of nuclei (blue channel) in SclCre^{ERT}; FoxM1^{fl/fl} mice 4 weeks after TAC operation with tamoxifen treatment. (scale bar, 10 μm). Arrows: endothelial cell nuclei; Arrowheads: myocardial cell nuclei.

FIG. 6L is a graph depicting quantitation of ventricle-body weight ratio in control and SclCre^{ERT}; FoxM1^{fl/fl} mice 4 weeks after sham or TAC operation. Ctrl: control hearts. Mut: SclCre^{ERT}; FoxM1^{fl/fl} hearts. P-value: Student's t-test. Error bar: SEM.

FIG. 6M is a micrograph depicting trichrome staining of cardiac fibrosis in control mice 4 weeks after sham or TAC operation. (scale bar, 50 μm).

FIG. 6N is a micrograph depicting trichrome staining of cardiac fibrosis in SclCre^{ERT}; FoxM1^{fl/fl} mice 4 weeks after sham or TAC operation. (scale bar, 50 μm).

FIG. 6O is a graph depicting echocardiographic measurement of fractional shortening of the left ventricle after 4 weeks of TAC.

FIG. 6P is a graph depicting quantitative RT-PCR analysis of ACE, ACE2 and ACE/ACE2 in the mice control and SclCre^{ERT}; FoxM1^{fl/fl} heart ventricles after sham or TAC operation. P-value: Student's t-test. Error bar: SEM.

FIG. 7A depicts quantitative PCR analysis of FoxM1 expression in normal and hypertrophic cardiomyopathy subjects (HCM) as analyzed in Example 5.

FIG. 7B depicts quantitative PCR analysis of ACE2/ACE ratio in normal and hypertrophic cardiomyopathy subjects (HCM) as analyzed in Example 5.

FIG. 7C is a fluorescence micrograph depicting an overlay of co-immunostaining images of Brg1 (red channel) and WGA (green channel) in heart of normal subjects (HCM). (scale bar, 10 μm). Arrows: endothelial cell; Arrowheads: myocardial cell.

FIG. 7D is a fluorescence micrograph depicting an overlay of co-immunostaining images of Brg1 (red channel) and WGA (green channel) in heart of hypertrophic cardiomyopathy subjects (HCM). (scale bar, 10 μm). Arrows: endothelial cell; Arrowheads: myocardial cell.

FIG. 7E is a fluorescence micrograph depicting an overlay of co-immunostaining images of FoxM1 (red channel) and WGA (green channel) in heart of normal subjects (HCM). (scale bar, 10 μm). Arrows: endothelial cell; Arrowheads: myocardial cell.

FIG. 7F is a fluorescence micrograph depicting an overlay of co-immunostaining images of FoxM1 (red channel) and WGA (green channel) in heart of hypertrophic cardiomyopathy subjects (HCM). (scale bar, 10 μm). Arrows: endothelial cell; Arrowheads: myocardial cell.

FIG. 7G depicts a working model of stress-induced FoxM1-Brg1 complex in the cardiac endothelial cells and ROS system in the heart.

FIG. 8A depicts expression of eNos, Et1, Adamts1, Hdac7, and Nrg1 in the stressed hearts as analyzed in Example 3.

FIG. 8B depicts immunostaining of heart ventricles as analyzed in Example 4.

FIG. 9A is a micrograph depicting measurement of cardiomyocyte size by wheat germ agglutinin (WGA) staining as analyzed in Example 4.

FIG. 9B is a micrograph depicting measurement of cardiomyocyte size by wheat germ agglutinin (WGA) staining as analyzed in Example 4.

FIG. 9C is a micrograph depicting measurement of cardiomyocyte size by wheat germ agglutinin (WGA) staining as analyzed in Example 4.

FIG. 9D is a micrograph depicting measurement of cardiomyocyte size by wheat germ agglutinin (WGA) staining as analyzed in Example 4.

FIG. 9E is a graph depicting measurement of cardiomyocyte size by wheat germ agglutinin (WGA) staining as analyzed in Example 4.

FIG. 10A is a Western blot depicting co-immunoprecipitation of Brg1 with FoxM1 in mice heart ventricles after 7 days TAC-operation.

FIG. 10B is a micrograph depicting a proximity ligation assay of Brg1-FoxM1 complex in nuclei of cultured mouse cardiac endothelial cells. IgG control: cells treated with IgG, but not primary anti-Brg1 or anti-FoxM1 antibodies.

FIG. 10C is a micrograph depicting a proximity ligation assay of Brg1-FoxM1 complex in nuclei of cultured mouse cardiac endothelial cells.

FIG. 10D is a graph depicting ChIP-qPCR analysis of ACE promoter using antibodies against FoxM1.

FIG. 10E is a graph depicting ChIP-qPCR analysis of ACE2 promoter using antibodies against FoxM1.

FIG. 10F is a graph depicting luciferase reporter assays of the ACE (−2983bp to +174bp) proximal promoter in MCEC cells. P-value: Student's t-test. Error bar: SEM.

FIG. 10G is a graph depicting luciferase reporter assays of the ACE2 (−7063bp to +786bp) proximal promoter in MCEC cells. P-value: Student's t-test. Error bar: SEM.

### DETAILED DESCRIPTION OF THE DISCLOSURE

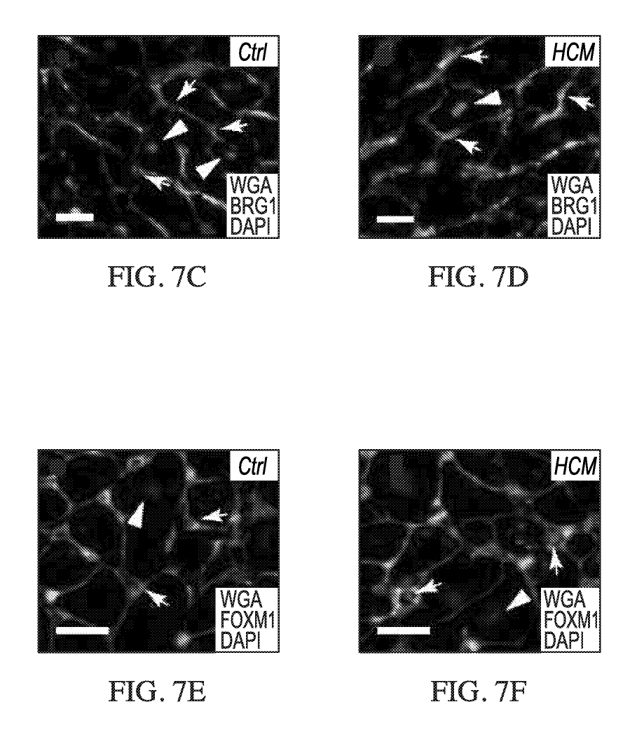

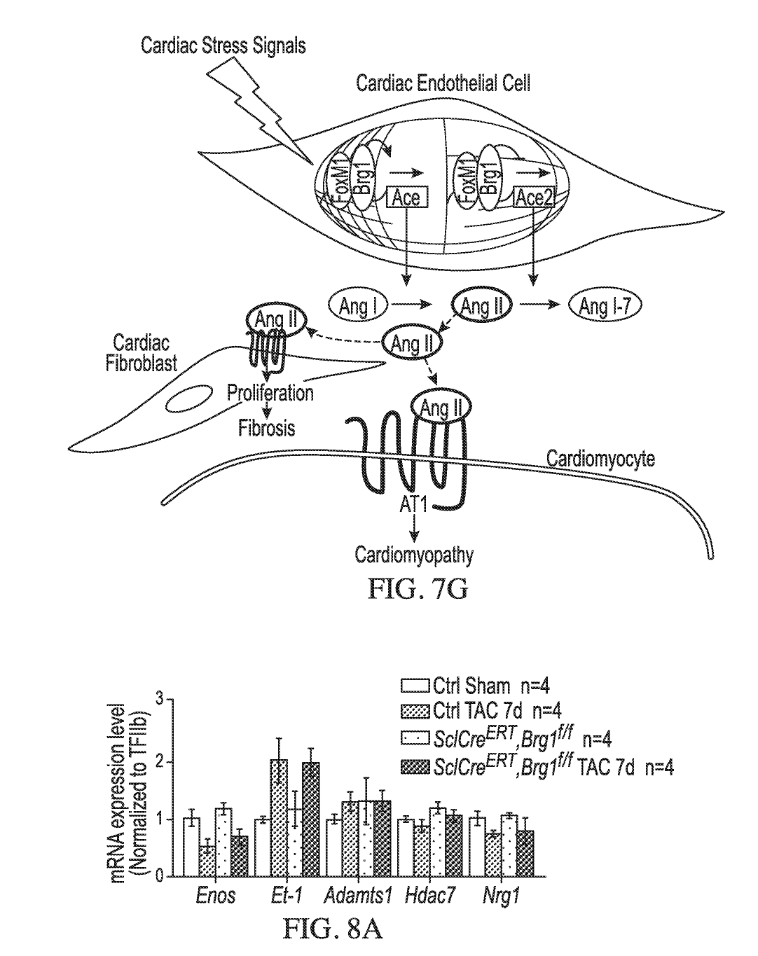

Controlling ACE/ACE2 expression is critical for maintaining cardiac function; increase of ACE or reduction of ACE2 is sufficient to cause cardiomyopathy. The present disclosure has now identified a new endothelial chromatin complex composed of Brg1 and FoxM1 that simultaneously activates ACE and represses ACE2 in response to cardiac stress (FIG. 7G). This provides new molecular insights into endothelial-myocardial interaction critical for heart function.

The present disclosure is directed to the use of a FoxM1 inhibitor, to prevent, reduce, and/or treat hypertrophy and heart failure. Particularly, in pathologically stressed hearts, FoxM1 and Brg1 are activated in cardiac endothelial cells. FoxM1 cooperates with Brg1 to activate angiotensin-converting enzyme (ACE) and inhibit angiotensin-converting enzyme (ACE2) expression, leading to increased production of angiotensin II, causing cardiac hypertrophy and failure. The present disclosure has found that a FoxM1 inhibitor can block the function of FoxM1-Brg1 complex, reversing the ACE/ACE2 expression ratio to protect the heart from hypertrophy and failure.

Suitable FoxM1 inhibitors include, for example, thiostrepton, SiomycinA, Forkhead Domain Inhibitor-6 (FDI-6), and combinations thereof. In one particular embodiment, the FoxM1 inhibitor is thiostrepton.

The FoxM1 inhibitor can be administered to a subject in need thereof to inhibit FoxM1 activation, thereby blocking the function of the FoxM1-Brg1 complex and reversing the ACE/ACE2 expression ratio. It has been found that such regulation of these pathways can provide protection of the heart from hypertrophy and failure. As used herein, “subject in need thereof” refers to a subset of subjects in need of treatment/protection from heart hypertrophy and/or failure. Some subjects that are in specific need of treatment may include subjects who are susceptible to, or at elevated risk of, experiencing heart hypertrophy and/or heart failure and symptoms of hypertrophy and/or failure. Subjects may be susceptible to, or at elevated risk of, experiencing symptoms of heart hypertrophy and/or heart failure due to family history, age, environment, and/or lifestyle. Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

Typically, the FoxM1 inhibitor is administered in an amount such to provide a therapeutically effective amount of the inhibitor to the subject. The term “therapeutically effective amount” as used herein, refers to that amount of active compound (i.e., FoxM1 inhibitor) or pharmaceutical agent that elicits the biological or medicinal response in a tissue system, animal or human that is being sought by a researcher, veterinarian, medical doctor or other clinician, which includes alleviation of the symptoms of the condition, disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the inhibitor described herein may be decided by the attending physician within the scope of sound medical judgment. The specific therapeutically-effective dose level for any particular subject will depend upon a variety of factors, including the condition, disease or disorder being treated and the severity of the condition, disease or disorder; activity of the specific inhibitor employed; the specific system employed; the age, body weight, general health, gender and diet of the subject: the time of administration, route of administration, and rate of excretion of the specific inhibitor employed; the duration of the treatment; drugs used in combination or coincidentally with the specific inhibitor employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of the inhibitor described herein. Further, it is appreciated that the co-therapies described herein may allow for the administration of lower doses of inhibitor that show such toxicity, or other undesirable side effect, where those lower doses are below thresholds of toxicity or lower in the therapeutic window than would otherwise be administered in the absence of a co-therapy.

In one embodiment, the FoxM1 inhibitor is administered in an amount of from about 5 mg/kg to about 20 mg/kg.

The term “administering” as used herein includes all means of introducing the FoxM1 inhibitor described herein to the subject, including, but are not limited to, oral (po), intravenous (iv), intramuscular (im), subcutaneous (sc), parenteral, transdermal, inhalation, buccal, ocular, sublingual, vaginal, rectal, and the like. The inhibitor described herein may be administered in unit dosage forms and/or formulations containing conventional nontoxic pharmaceutically-acceptable carriers, adjuvants, and vehicles.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustratively, administering includes local use, such as when administered locally to the site of disease, injury, or defect, or to a particular organ or tissue system. Illustrative local administration may be performed during open surgery, or other procedures when the site of disease, injury, or defect is accessible. Alternatively, local administration may be performed using parenteral delivery where the inhibitor described herein is deposited locally to the site without general distribution to multiple other non-target sites in the subject being treated. It is further appreciated that local administration may be directly in the injury site, or locally in the surrounding tissue. Similar variations regarding local delivery to particular tissue types, such as organs, and the like, are also described herein.

In some embodiments, a therapeutically effective amount of FoxM1 inhibitor in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the inhibitor can be administered in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The FoxM1 inhibitor-containing formulations may contain anywhere from about 0.1% by weight to about 99.9% by weight active ingredients, depending upon the selected dose and dosage form.

The following examples further illustrate specific embodiments of the present disclosure; however, the following illustrative examples should not be interpreted in any way to limit the disclosure.

### EXAMPLES

### Example 1

In this Example, endothelial factors that are mis-regulated by cardiac pressure stress were analyzed.

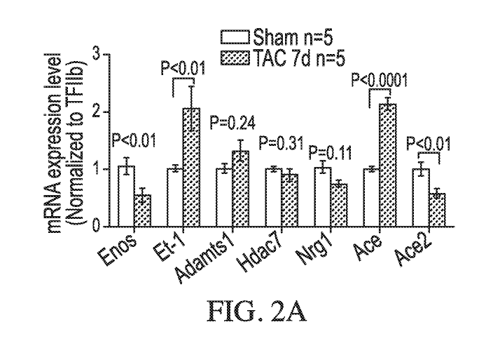

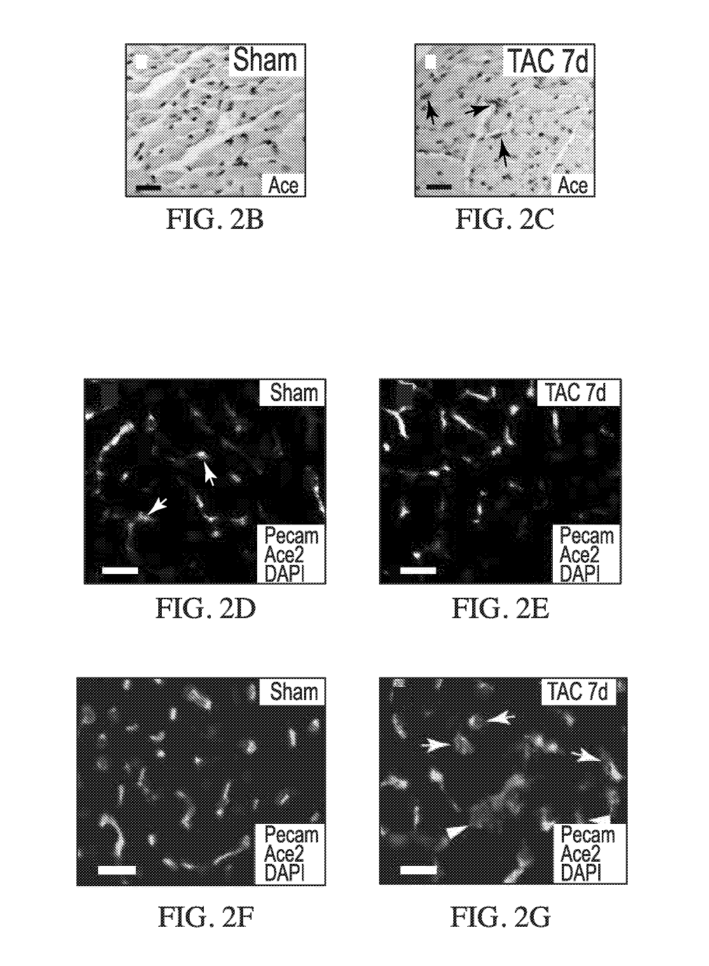

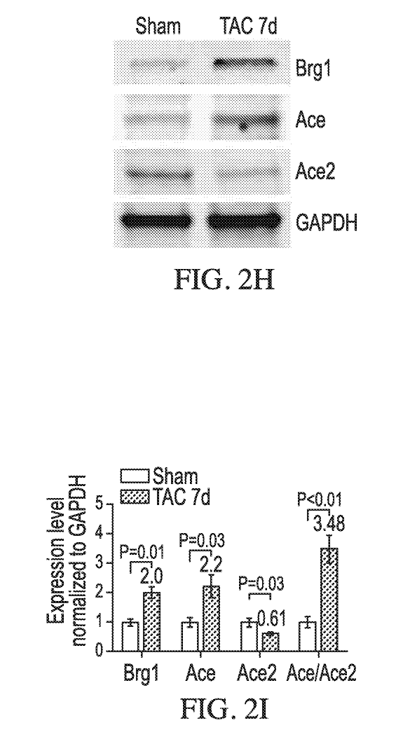

Particularly, by using reverse transcription and quantitative polymerase chain reaction (RT-qPCR), the expression of cardiac endothelial factors in the left ventricle with or without transaortic constriction (TAC) were examined. These factors included eNos, Et-1, Adamts1, Hdac7, Nrg1, ACE and ACE29. Within 7 days after TAC, Et-1 and ACE were induced 2.0- and 2.9-fold in left ventricles, whereas Enos and ACE2 were reduced by 46% and 48% (FIG. 2A). Adamts1, Hdac7, and Nrg1 had no significant changes. The regulation of ACE and ACE2, which encode secreted enzymes that counter each other to regulate the amount of angiotensin 2 that is critical for cardiovascular function, were focused on.

Prior to the present disclosure, it was unknown how ACE and ACE2 were regulated in the heart. Using immunostaining to further assess the regulation of ACE and ACE2 by cardiac stress, it was found that ACE was activated by TAC, whereas ACE2 was suppressed in endothelial cells of the heart (FIGS. 2B-2E). Particularly, immunostaining showed that Ace proteins were present at low levels in healthy hearts but up-regulated in the endothelium of stressed hearts (FIGS. 2B and 2C). In contrast, Ace2 proteins were present at high levels in the endothelium of healthy hearts, but down-regulated in TAC-stressed hearts (FIGS. 2D and 2E). Western blot analysis of the stressed hearts confirmed that Ace proteins were up-regulated to 2.2-fold and Ace2 proteins reduced by 0.49-fold, with the ratio of Ace/Ace2 proteins changed by 3.48-fold in the stressed hearts (FIGS. 2H and 2I).

In view that Ace is known to promote cardiac pathology, whereas Ace2 inhibits cardiomyopathy, such opposite expression dynamics indicate that a loss of balance between Ace and Ace2 in pressure-stressed hearts is crucial for pathological hypertrophy. Furthermore, the magnitude of stress-induced changes of Ace and Ace2 proteins was comparable to that of mRNA (FIGS. 2A and 2I), indicating that the primary regulation of Ace and Ace2 in stressed hearts occurs at the transcription level. Because Ace is known to promote cardiac pathology, and Ace2 inhibits cardiac pathology, such opposite expression dynamics indicated that a loss of balance between the pathogenic Ace and the cardioprotective Ace2 in pressure-stressed hearts is crucial for pathological hypertrophy.

### Example 2

In this Example, the antithetical regulation of ACE and ACE2 in the endothelium of stressed hearts was examined.

One important mechanism of gene regulation is through chromatin remodeling. By immunostaining, it was observed that Brg1, a crucial ATP-dependent chromatin-remodeling factor, was expressed at a low level in endothelial cells of healthy adult hearts (FIG. 2F). However, the expression of Brg1 was highly activated by TAC in cardiomyocytes and cardiac endothelial cells (FIGS. 2G, 2H, and 2I). It was previously shown that activation of Brg1 in cardiomyocytes is essential for cardiomyopathy to develop (Hang et al., “Chromotin regulation by Brg1 underlies heart muscle development and disease,” Nature 466, 62-67 (2010); Han et al., “A long non-coding RNA protects the heart from pathology hypertrophy,” Nature in Press (2014)), but the role of stress-activated Brg1 in cardiac endothelial cells remains unknown.

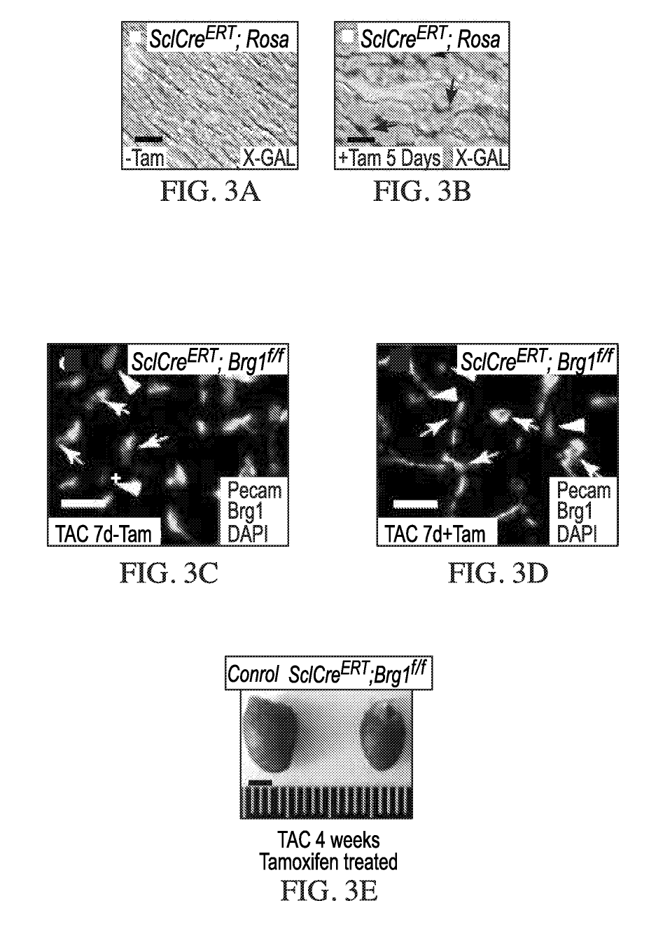

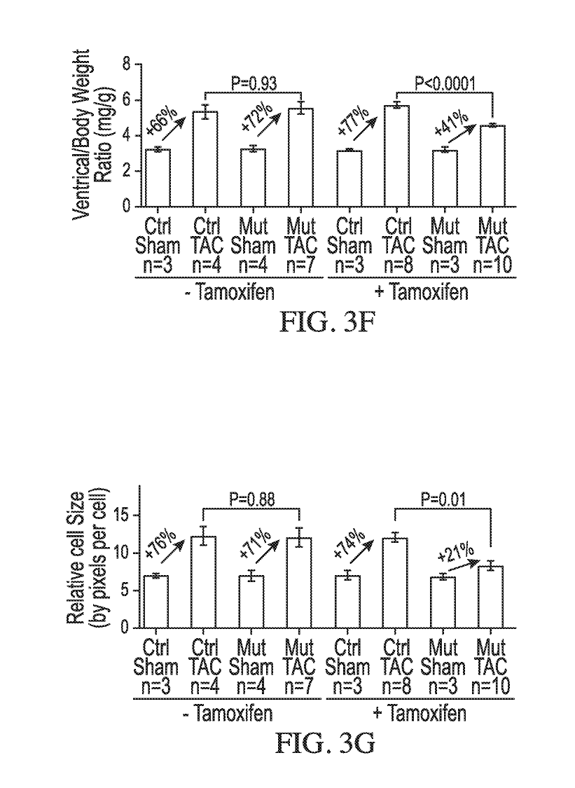

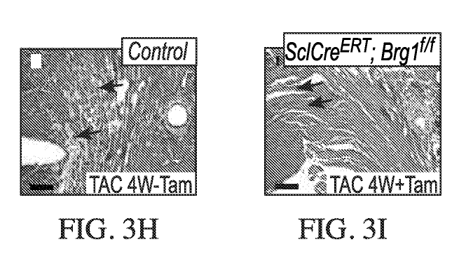

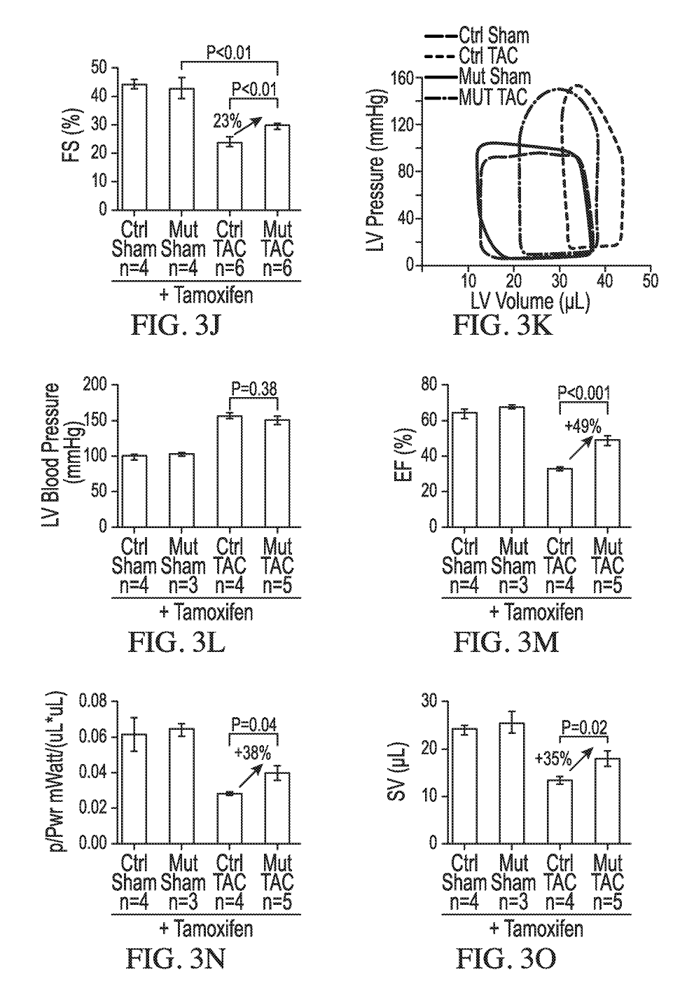

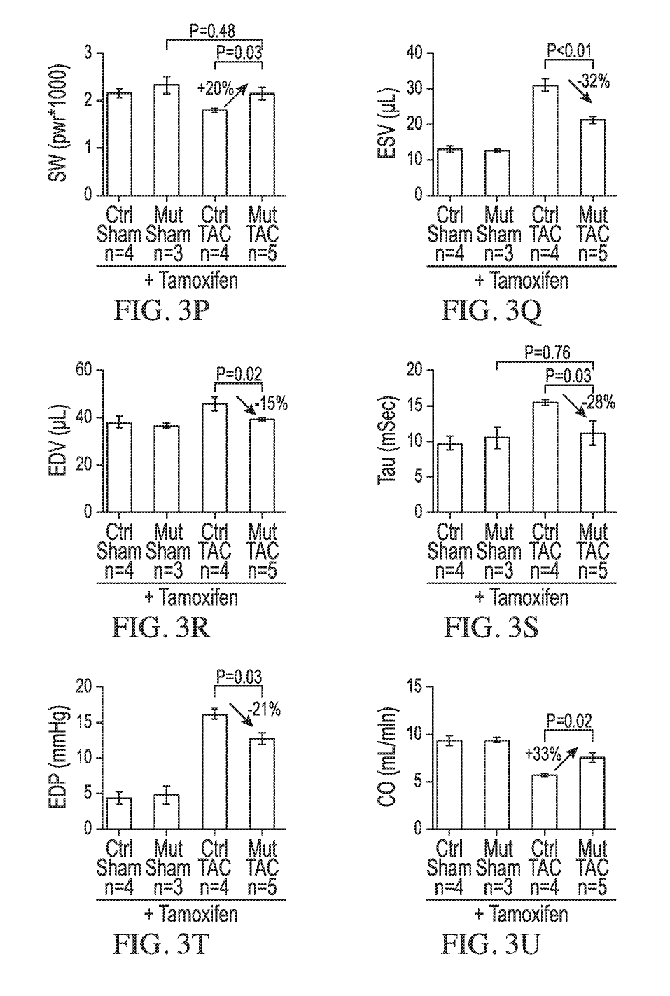

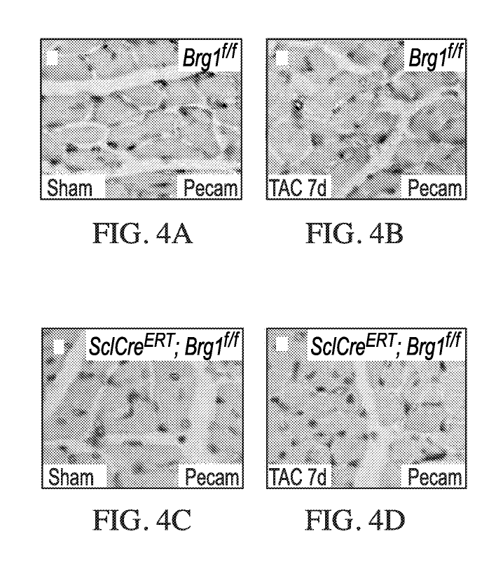

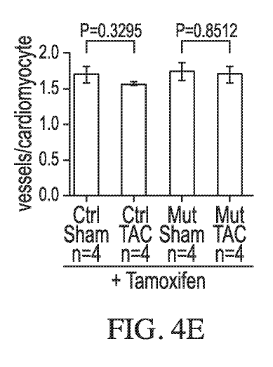

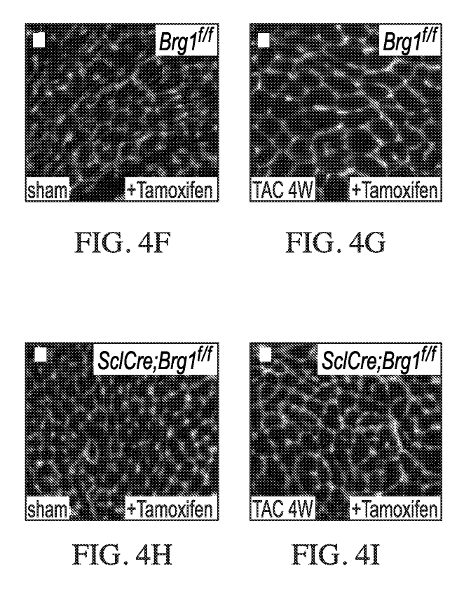

Given that Brg1 represses α-MHC (Myh6) and activates β-MHC (Myh7) to trigger MHC switch in cardiomyocytes of stressed hearts, it was hypothesized that Brg1 could also control the antithetical expression of ACE and ACE2 in the endothelium of stressed hearts to trigger myopathy. To test this hypothesis, it was determined if endothelial Brg1 was essential for cardiac hypertrophy. A tamoxifen-dependent SclCre^{ERT} mouse line was used to induce endothelial Brg1 deletion in mice that carried floxed alleles of Brg1 gene (Brg1^{f}). By immunostaining, it was shown that tamoxifen treatment for 5 days (0.1 mg/g body weight, oral gavage once every other day, 3 doses total) before the TAC surgery was sufficient to activate a β-galactosidase reporter (FIGS. 3A and 3B) and to disrupt Brg1 activation in the endothelial cells, but not cardiomyocytes, in stressed hearts (FIGS. 3C, 3D). A TAC procedure was then performed to pressure-overload the heart and induce cardiac hypertrophy in the control and SclCre^{ERT}; Brg1^{f/f} littermate mice with or without tamoxifen treatment. Four weeks after TAC, the control mice had larger hearts than SclCre^{ERT}; Brg1^{f/f} mice that lacked endothelial Brg1 (FIG. 3E). Analysis of the cardiac mass (ventricular weight/body weight ratio) showed an approximately 50 percent reduction (from 77% to 41%) of cardiac hypertrophy in SclCre^{ERT}; Brg1^{f/f} mice (FIG. 3F). Measurement of cardiomyocyte size by wheat germ agglutinin (WGA) staining (FIGS. 4F-4I) revealed an approximately 70 percent reduction (from 74% to 21%) of cardiomyocyte size in SclCre^{ERT}; Brg1^{f/f} mice. Also there was a dramatic reduction of interstitial fibrosis in the SclCre^{ERT}; Brg1^{f/f} mice (FIGS. 3H and 3I). Furthermore, within four weeks after TAC, SclCre^{ERT}; Brg1^{f/f} mice showed 23% improvement of left ventricular fractional shortening (FS) by echocardiography (P<0.01)(FIG. 3J).

To further determine cardiac function, a catheter was inserted into the left ventricle (LV) to measure its LV pressure and volume at any instant of the cardiac cycle (FIG. 3K). The in vivo catheterization showed a peak pressure overload of ˜50 mmHg, with TAC increasing peak LV systolic pressure from 100 to 150 mmHg (FIG. 3L), and the pressure load was comparable between control and SclCre^{ERT}; Brg1^{f/f} mice (FIG. 3L). It was found that endothelial Brg1 deletion greatly improved the function of TAC-stressed hearts. SclCre^{ERT}; Brg1^{f/f} mice exhibited much better cardiac function four weeks after TAC. Ejection fraction (EF) improved by 49% (p<0.001) (FIG. 3M), preload-adjusted maximal power (plPwr) by 38% (p=0.04) (FIG. 3N), stroke volume (SV) by 35% (p=0.02) (FIG. 3O), and stroke work (SW) by 20% (p=0.03) (FIG. 3P). Also, SclCre^{ERT}; Brg1^{f/f} mice had less dilated hearts, with end systolic volume (ESV) reduced by 32% (p<0.01) (FIG. 3Q) and end diastolic volume (EDV) reduced by 15% (p=0.02) and normalized (FIG. 3R). Both the LV contractility and volume measurement indicated a major improvement in systolic function of the heart. On the other hand, SclCre^{ERT}; Brg1^{f/f} mice had improved diastolic function. This was evidenced by the reduction of isovolumic relaxation time constant Tau by 42.3% (p=0.01) (FIG. 3S), and end diastolic pressure (EDP) by 21% (p=0.03) (FIG. 3T). By improving systolic and diastolic function of the heart, SclCre^{ERT}; Brg1^{f/f} mice showed 33% (P=0.02) increase of cardiac output (CO)(FIG. 3U). Overall, endothelial Brg1-null mice had a 50-70% reduction of cardiac hypertrophy, minimal/absent cardiac fibrosis, and great increase of cardiac function after TAC. These findings indicate that the Brg1 is activated by stress in cardiac endothelial cells to trigger myopathy.

### Example 3

In this Example, as angiogenesis underlies cardiac hypertrophy and failure, cardiac vessel density was examined to test if endothelial Brg1 was essential for vascular supply in stressed hearts. By PECAM staining, no difference was found in the vessel density of control and SclCre^{ERT}; Brg1^{f/f} hearts treated with tamoxifen and TAC (FIGS. 4A-4D). This suggests that endothelial Brg1 does not regulate cardiac hypertrophy through angiogenesis.

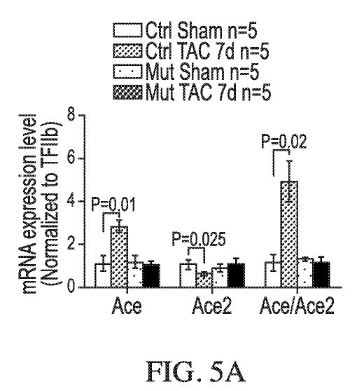

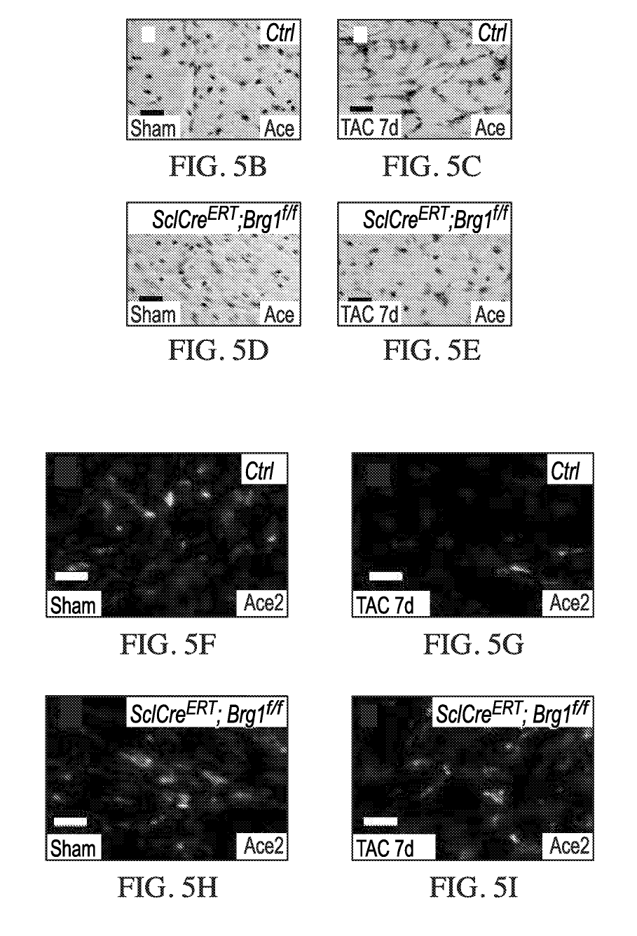

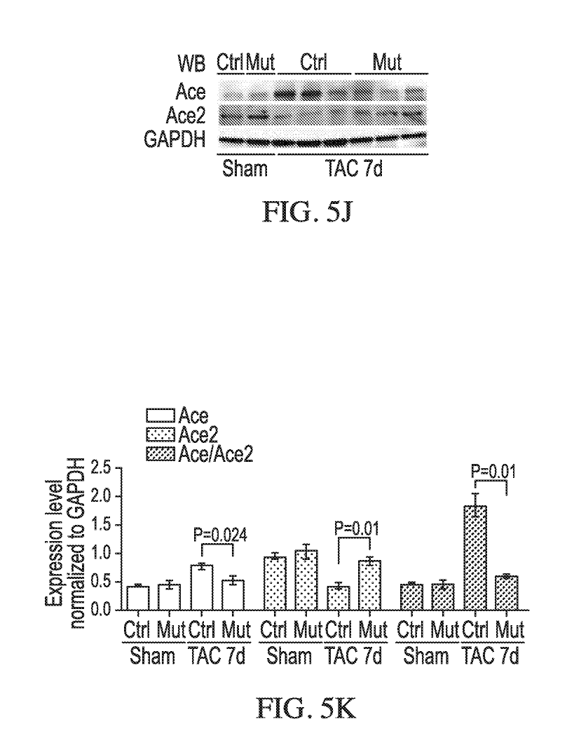

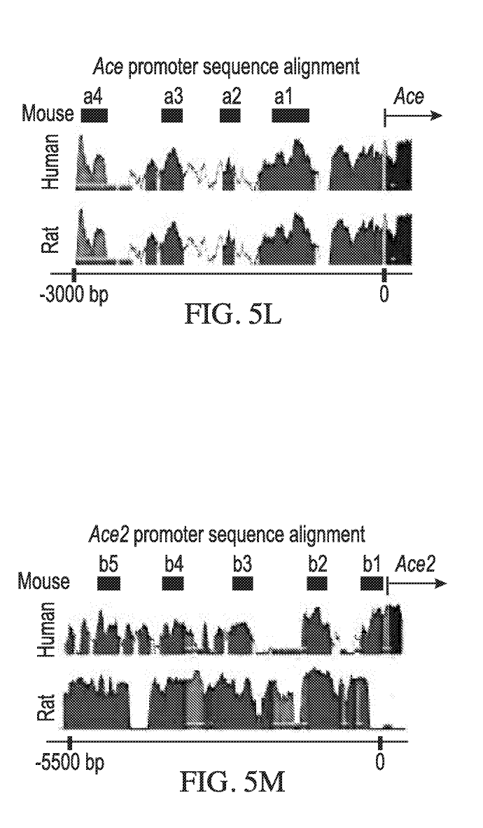

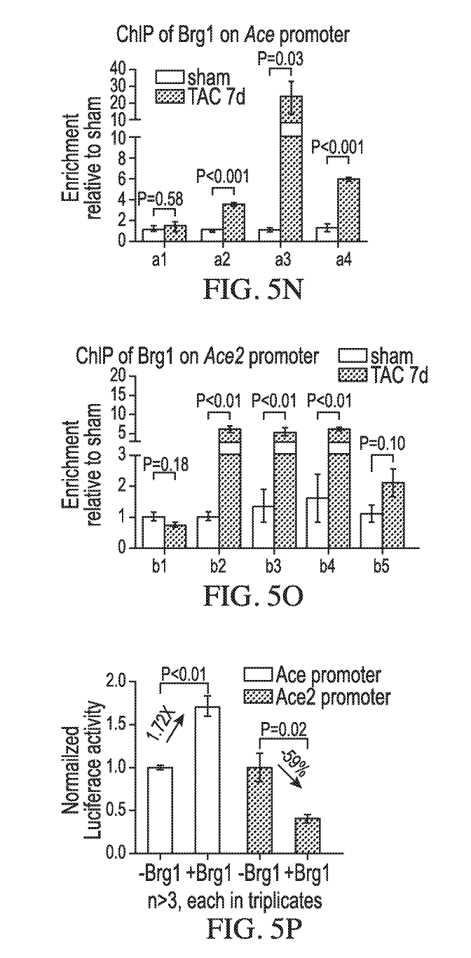

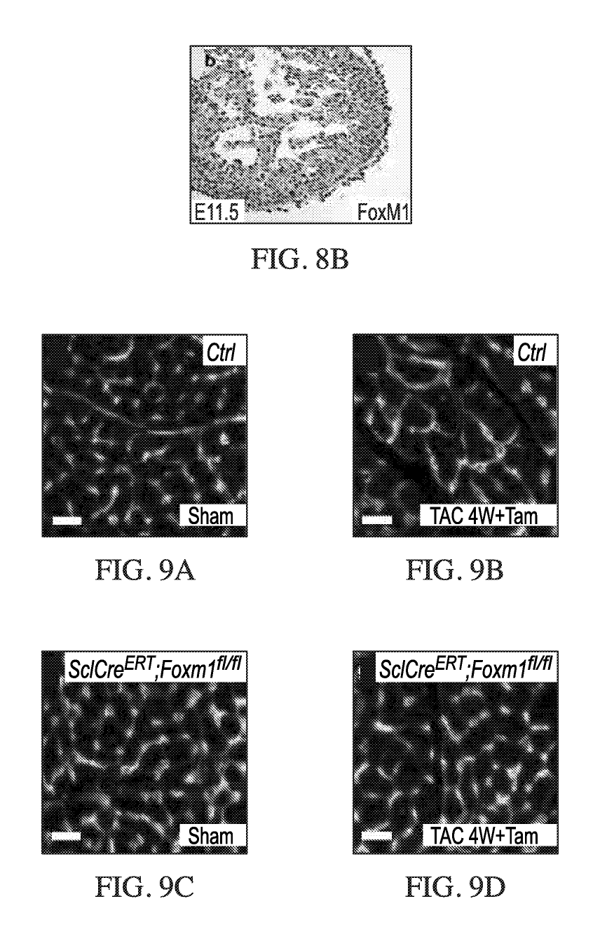

Given the role of Ace and Ace 2 in cardiomyopathy, endothelial Brg1 was tested to determine if it was essential for the dynamic changes of Ace and Ace2 in stressed hearts. By RT-qPCR, the expression of eNos, Et1, Adamts1, Hdac7, Nrg1, Ace and Ace 2 was examined in tamoxifen-treated control and SclCre^{ERT}; Brg1^{f/f} hearts with or without TAC. Among these genes and after 7 days of TAC, the opposite changes of Ace and Ace 2 were evident in the stressed hearts of control mice, with TAC increasing Ace/Ace2 ratio by 4.5-fold (FIG. 5A). However, these changes of Ace and Ace2 were eliminated in TAC-stressed hearts of SclCre^{ERT}; Brg1^{f/f} mice (FIG. 5A), indicating that endothelial Brg1 is essential for the stress-induced changes of Ace/Ace2 in the hearts. In contrast, the changes of other endothelial genes (Et1, Enos, Adamts1, Hdac7, Nrg1) in the stressed hearts were not affected by endothelial Brg1 (FIG. 8A), suggesting a certain degree of Brg1 specificity in the control of Ace/Ace2 pathological switch Immunostaining revealed that Ace and Ace2 proteins were present in the endothelium of control hearts (FIGS. 5B and 5E), with Ace up-regulated and Ace2 down-regulated by TAC (FIGS. 5B and 5C, FIGS. 5F and 5G). In contrast, TAC-induced up-regulation of Ace and down-regulation of Ace2 proteins were greatly reduced or abolished in TAC-stressed SclCre^{ERT}; Brg1^{f/f} hearts (FIGS. 5B-5I). These findings using RT-qPCR and immunostaining were confirmed by Western blot analysis of Ace and Ace2 using the left ventricular protein extracts from the control and mutant mice (FIGS. 5J and 5K). Collectively, the results indicate that endothelial Brg1 is activated by cardiac stress to disturb the homeostasis of pro-myopathic Ace and anti-myopathic Ace2, resulting in cardiac hypertrophy and failure.

To determine if Brg1 directly regulated the expression of Ace and Ace2 in the stressed hearts, the binding of Brg1 to the Ace and Ace2 promoters was examined. With sequence alignment, four regions (a1-a4) were identified in the ˜3 Kb upstream region of the mouse Ace promoter that are evolutionarily conserved in mouse, rat and human (FIG. 5I). Chromatin immunoprecipitation (ChIP) assay using anti-Brg1 antibody showed that in the TAC-operated hearts Brg1 was highly enriched in three of a1-a4 regions (a2, a3, and a4), compared to the sham-operated hearts (FIG. 5N). Additionally, the 5.5 kb upstream region of the mouse Ace2 promoter, which contained five highly conserved regions among different species (b1-b5 in FIG. 5M), were analyzed. ChIP analysis of the TAC-stressed heart ventricles showed that Brg1 was highly enriched in three of the b1-b5 regions (b2, b3, and b4), compared to the sham-operated hearts (FIG. 5O). These ChIP studies of stressed hearts reveal that once activated by stress, Brg1 binds to evolutionarily conserved regions of Ace and Ace2 promoters.

The transcriptional activity of Brg1 on the Ace and Ace2 promoters was also tested. 3.1 kb of Ace upstream promoter (−2983bp to +174bp) and 7.8 Kb of Ace2 upstream promoter (−7063bp to +786bp) were cloned into the episomal reporter pREP4 that undergoes chromatinization in mammalian cells. The reporter constructs and Brg1-expressing plasmid were transfected into mouse cardiac endothelial cells. In these cells, Brg1 caused a 1.7-fold increase in Ace promoter activity and 59% reduction in Ace2 promoter activity (FIG. 5P). Combined with the ChIP results, these reporter studies indicate that Brg1 activates Ace promoter and represses Ace2 promoter, providing a mechanism for the antithetical changes of Ace and Ace2 in stressed hearts.

### Example 4

In this Example, the activity of FoxM1 in fetal hearts was analyzed.

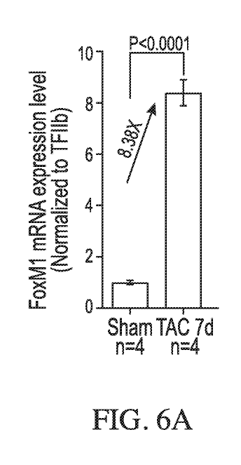

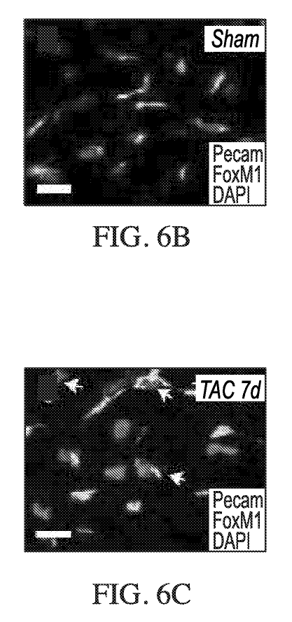

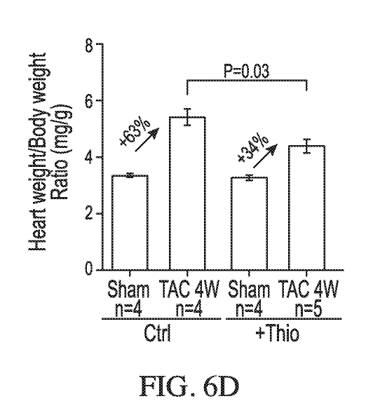

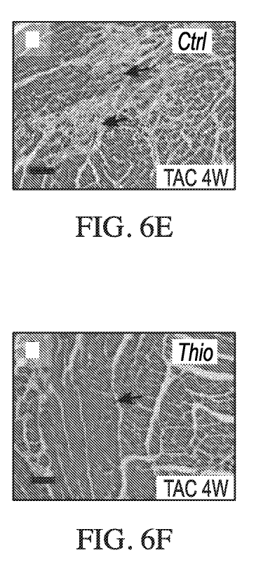

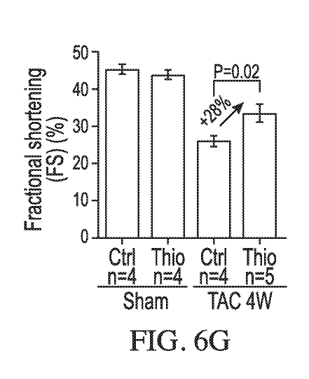

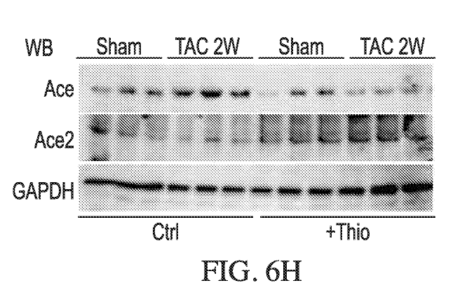

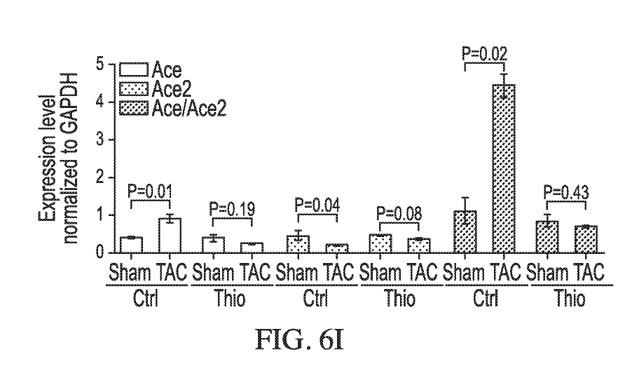

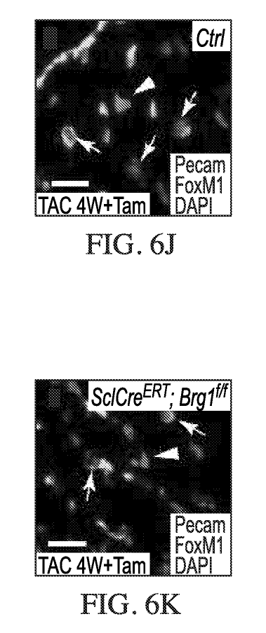

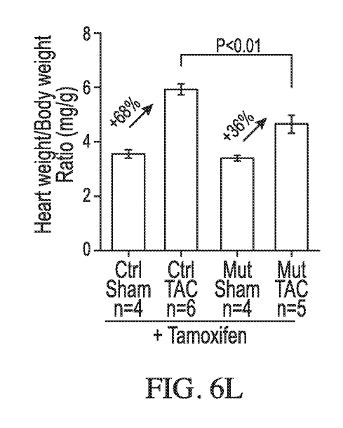

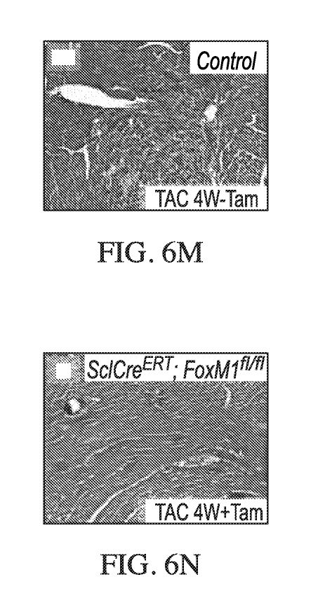

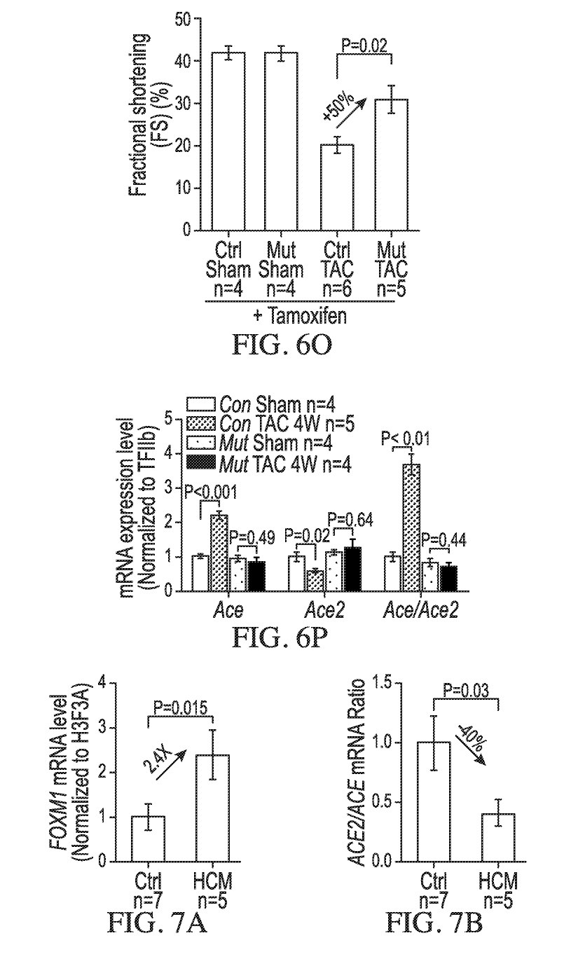

FoxM1 is a transcription factor that regulates the expression of genes associated with pathological hypertrophy. By RT-qPCR and immunostaining of heart ventricles, it was found that FoxM1 was abundant in the fetal hearts (FIG. 8B), but was down-regulated in the adult hearts. However, FoxM1 mRNA increased by 8.4-fold in TAC-stressed hearts (FIG. 6A), and the protein was present in the nuclei of both myocytes and endothelial cells of stressed hearts (FIGS. 6B and 6C). Because of the stress-induced endothelial expression of FoxM1, it was evaluated if FoxM1 cooperated with Brg1 to regulate Ace and Ace2 expression. The necessity of FoxM1 activation for cardiac hypertrophy by using FoxM1 inhibitor thiostrepton to inhibit FoxM1 in TAC-stressed hearts was tested. Within 4 weeks after TAC, the control mice injected with DMSO developed severe cardiac hypertrophy with increased ventricle—body weight ratio, interstitial fibrosis, and cardiac dysfunction with reduced left ventricular fractional shortening (FS) (FIGS. 6D, 6E, and 6G). In contrast, thiostrepton-treated mice exhibited mild cardiac hypertrophy, mild interstitial fibrosis (FIGS. 6D and 6F) and a lesser degree of cardiac dysfunction (FIG. 6G). There was a ˜50% reduction of hypertrophy and 28% improvement of FS, comparable to the changes observed in endothelial Brg1-null hearts (FIGS. 3F and 3J). In addition, Western blot analysis of heart ventricles showed that the TAC-induced changes of Ace and Ace2 were abolished when FoxM1 was inhibited by thiostrepton (FIGS. 6H and 6I). Thiostrepton reduced Ace/Ace2 ratio in stressed hearts by 6.53-fold (FIG. 6I). Collectively, these findings indicate that FoxM1 activation by stress is necessary for cardiac myopathy and for stress-induced changes of Ace and Ace2.

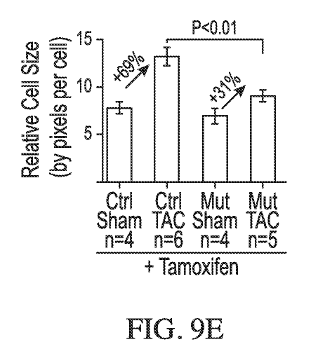

A genetic method was also used to delete FoxM1 in endothelial cells. By crossing tamoxifen-dependent SclCreER mouse line (Gothert, J. R., et al., Blood 104, 1769-1777 (2004)) with the mice that carried floxed alleles of FoxM1 gene, FoxM1 activation was disrupted in the endothelial cells, but not cardiomyocytes, in TAC stressed hearts (FIGS. 6J and 6K). Four weeks after TAC, analysis of the cardiac mass (ventricular weight/body weight ratio) showed an approximately 50 percent reduction (from 68% to 36%) of cardiac hypertrophy in SclCreERT; FoxM1fl/fl mice (FIG. 6L). Measurement of cardiomyocyte size by wheat germ agglutinin (WGA) staining (FIGS. 9A-9D) revealed an approximately 55 percent reduction (from 69% to 31%) of cardiomyocyte size in SclCreERT; FoxM1fl/fl mice (FIG. 9E). Also there was a dramatic reduction of interstitial fibrosis in the SclCreERT; FoxM1fl/fl mice (FIGS. 6M and 6N). Furthermore, within four weeks after TAC, SclCreERT; FoxM1fl/fl mice showed 50% improvement of left ventricular fractional shortening (FS) by echocardiography (P=0.02) (FIG. 60). Also, real-time PCR analysis of heart ventricles showed that the TAC-induced changes of Ace and Ace2 were abolished when FoxM1 was deleted in endothelial cells (FIG. 6P).

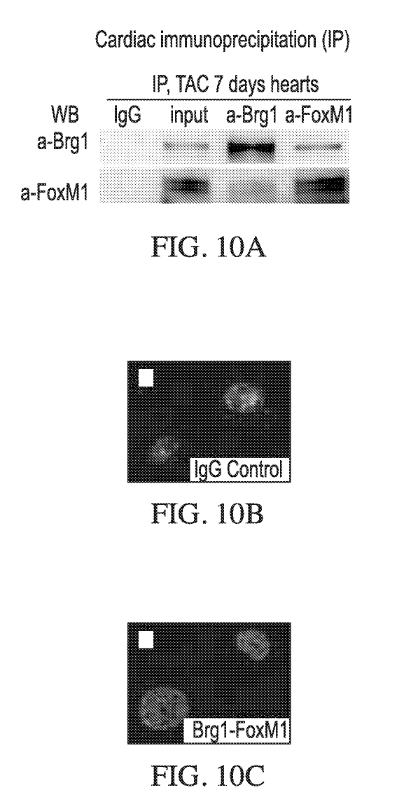

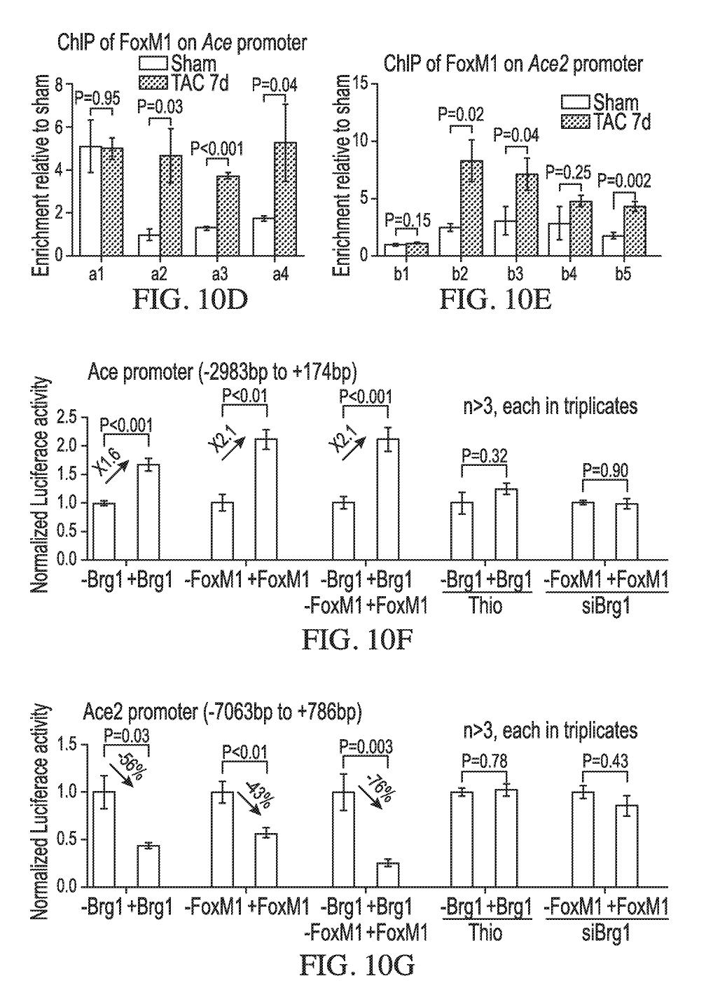

Because both Brg1 and FoxM1 were stress-activated factors essential for cardiac hypertrophy and ACE/ACE2 regulation, it was examined whether Brg1 and FoxM1 could form a physical complex to control gene expression. Co-immunoprecipitation studies of heart ventricles showed that Brg1 co-immunoprecipitated with FoxM1 in the stressed hearts (FIG. 10A). Proximity ligation (Duolink) assay further showed that Brg1 and FoxM1 formed a protein complex in the nuclei of mouse cardiac endothelial cells (FIGS. 10A and 10B). It was then analyzed whether FoxM1 could bind to the promoters of Ace and Ace2 in the stressed hearts. ChIP analysis of TAC-treated hearts showed that FoxM1 was highly enriched in the conserved regions of Ace and Ace 2 promoters relative to the sham-operated hearts (FIGS. 10B and 10C). Using this assay, it was shown that Brg1 and FoxM1 did form a complex in cultured mouse cardiac endothelial cells (FIGS. 10B and 10C). It was then determined if FoxM1 could bind to the promoters of Ace and Ace2 in the stressed hearts. ChIP analysis of TAC-treated hearts showed that FoxM1 was highly enriched in the conserved regions of Ace and Ace2 promoters relative to the sham-operated hearts (FIGS. 10D and 10E). The binding pattern of FoxM1 to a1-a4 regions of Ace and to b1-b5 regions of Ace2 was similar to that of Brg1 (FIGS. 5N and 5O). The ChIP analyses, combined with the existence of stress-induced Brg1-FoxM1 complex (FIGS. 10A and 10C), suggest that Brg1 and FoxM1 cooperate to regulate the dynamic expression of Ace and Ace2 in the stressed hearts.

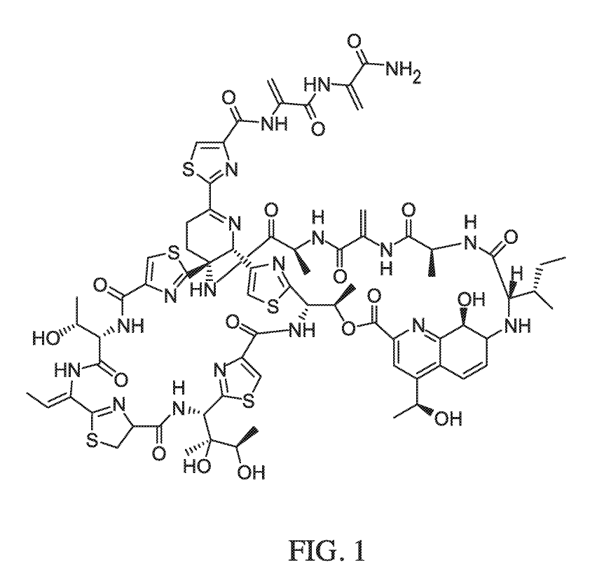

Consistently with the ChIP results, luciferase reporter assays conducted in mouse cardiac endothelial cells showed that FoxM1, like Brg1, was capable of activating Ace and repressing Ace2 promoter activities (FIGS. 1OF and 10G). Inhibition of FoxM1 by thiostrepton eliminated the ability of Brg1 to activate Ace and repress Ace2 promoter (FIGS. 10F and 10G). Likewise, knockdown of Brg1 abolished FoxM1's activity on Ace activation and Ace2 repression (FIGS. 10F and 10G), suggesting that Brg1 and FoxM1 are mutually dependent for the regulation of Ace and Ace2 promoters. Overall, the ChIP and reporter analyses, combined with the presence of stress-induced Brg1-FoxM1 complex, suggested that Brg1 and FoxM1 cooperate to regulate the pathological switch of Ace and Ace2 in the stressed hearts.

### Example 5

In this Example, it was examined whether Brg1 and FoxM1 were also activated in cardiac endothelial cells of human hypertrophic hearts. Particularly, subjects with left ventricular hypertrophy (LVH) were studied.

The tissue samples were obtained from donor hearts that were considered unsuitable for transplantation due to the lack of timely recipients or mismatched surgical cut. RT-qPCR analysis showed that hearts with LVH had a 2.4-fold increase of FoxM1 and 40% reduction of Ace2/Ace expression (FIGS. 7A and 7B) Immunostaining showed that both Brg1 and FoxM1 were activated in both myocytes and endothelial cells of the hypertrophic hearts (FIGS. 7C-7F), similar to those seen in stressed mouse hearts. This suggests a conserved mechanism underlying myopathy of mouse and human hearts.

In summary, the requirement of Brg1-FoxM1 complex for myopathy to develop has important implications for heart failure therapy. In stressed hearts, FoxM1 chemical inhibitor was effective in reversing Ace/Ace2 and preventing myopathy, indicating that concurrently pharmacologically inhibiting ACE and activating Ace2 improves heart function of patients with heart failure. Although Ace inhibitors are clinically available, there has not been any chemical activator of Ace2, likely due to the difficulty of generating an Ace2 protein activator of any kind. In this regard, the chemical inhibition of Brg1-FoxM1 complex is particularly salient for heart failure therapy and provides a new pharmacological method that simultaneously targets Ace and Ace2 genes to reverse the Ace/Ace2 ratio in failing hearts.

This written description uses examples to disclose the invention and also to enable any person skilled in the art to practice the invention, including performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for treating cardiac hypertrophy in a subject in need thereof, the method comprising administering to the subject a FoxM1 inhibitor.

2. The method as set forth in claim 1 wherein the FoxM1 inhibitor is selected from the group consisting of thiostrepton, siomycinA, Forkhead Domain Inhibitor-6 (FDI-6), and combinations thereof.

3. The method as set forth in claim 1 wherein the FoxM1 inhibitor is thiostrepton.

4. The method of claim 1 wherein the FoxM1 inhibitor is administered using an administration route selected from the group consisting of: oral (po), intravenous (iv), intramuscular (im), subcutaneous (sc), parenteral, transdermal, inhalation, buccal, ocular, sublingual, vaginal, rectal, and combinations thereof.

5. The method of claim 1 wherein the FoxM1 inhibitor is administered in a formulation further comprising at least one excipient or carrier.

6. (canceled)

7. The method of claim 5 wherein the FoxM1 inhibitor is administered in a form selected from the group consisting of tablet, pill, powder, lozenge, sachet, cachet, elixir, suspension, emulsion, solution, syrup, aerosol, ointment, gelatin capsule, suppository, sterile injectable solution, and sterile packaged powder.

8. A method for treating cardiac failure in a subject in need thereof, the method comprising administering to the subject a FoxM1 inhibitor.

9. The method as set forth in claim 8 wherein the FoxM1 inhibitor is selected from the group consisting of thiostrepton, siomycinA, Forkhead Domain Inhibitor-6 (FDI-6), and combinations thereof.

10. The method as set forth in claim 8 wherein the FoxM1 inhibitor is thio strepton.

11. The method of claim 8 wherein the FoxM1 inhibitor is administered using an administration route selected from the group consisting of: oral (po), intravenous (iv), intramuscular (im), subcutaneous (sc), parenteral, transdermal, inhalation, buccal, ocular, sublingual, vaginal, rectal, and combinations thereof.

12. The method of claim 8 wherein the FoxM1 inhibitor is administered in a formulation further comprising at least one excipient or carrier.

13. (canceled)

14. The method of claim 12 wherein the FoxM1 inhibitor is administered in a form selected from the group consisting of tablet, pill, powder, lozenge, sachet, cachet, elixir, suspension, emulsion, solution, syrup, aerosol, ointment, gelatin capsule, suppository, sterile injectable solution, and sterile packaged powder.

15. A method of modulating ACE/ACE2 enzyme ratio in a subject in need thereof, the method comprising administering to the subject a FoxM1 inhibitor.

16. The method as set forth in claim 15 wherein the FoxM1 inhibitor is selected from the group consisting of thiostrepton, siomycinA, Forkhead Domain Inhibitor-6 (FDI-6), and combinations thereof.

17. The method of claim 15 wherein the FoxM1 inhibitor is administered using an administration route selected from the group consisting of: oral (po), intravenous (iv), intramuscular (im), subcutaneous (sc), parenteral, transdermal, inhalation, buccal, ocular, sublingual, vaginal, rectal, and combinations thereof.

18. The method of claim 15 wherein the FoxM1 inhibitor is administered in a formulation further comprising at least one excipient or carrier.

19. (canceled)

20. The method of claim 18 wherein the FoxM1 inhibitor is administered in a form selected from the group consisting of tablet, pill, powder, lozenge, sachet, cachet, elixir, suspension, emulsion, solution, syrup, aerosol, ointment, gelatin capsule, suppository, sterile injectable solution, and sterile packaged powder.

21. The method of claim 1 wherein the subject is administered the FoxM1 inhibitor in an amount of from about 5 mg/kg to about 20 mg/kg.

22. The method of claim 8 wherein the subject is administered the FoxM1 inhibitor in an amount of from about 5 mg/kg to about 20 mg/kg.

23. The method of claim 15 wherein the subject is administered the FoxM1 inhibitor in an amount of from about 5 mg/kg to about 20 mg/kg.
